(19) United States
(12) Patent Application Publication
(10) Pub. No.: US 2017/0333546 A1
(43) Pub. Date: Nov. 23, 2017

(54) Method for Producing and Vaccine Composition of Neisseria Meningitidis Serogroups A, C, Y, and W-135 Oligosaccharides Conjugated to Glycan-Free Carrier Protein

(71) Applicant: Jl Medical Corporation, Omaha, NE (US)

(72) Inventor: Jeeri R. Reddy, Omaha, NE (US)

(21) Appl. No.: 15/370,983

(22) Filed: Dec. 6, 2016

Related U.S. Application Data
(60) Provisional application No. 62/264,755, filed on Dec. 8, 2015.

(51) Int. Cl. A61K 39/095; A61K 39/00

(52) U.S. Cl. CPC A61K 39/095; A61K 2039/70; A61K 2039/55; A61K 2039/6037

(57) ABSTRACT

A multivalent vaccine composition, a method of producing the multivalent vaccine composition, and an apparatus containing the multivalent vaccine composition. The multivalent vaccine composition may include a mixture. The mixture may include Neisseria meningitides serogroups A, C, Y, and W-135 oligosaccharides conjugated to glycan-free carrier proteins. When administered, the multivalent vaccine composition may provide long-lasting immunity to humans of all age groups, including infants.

### CROSS-REFERENCE TO RELATED APPLICATIONS

This application claims the benefit of U.S. Provisional Application No. 62/264,755, filed on Dec. 8, 2015, which is hereby expressly incorporated herein in its entirety.

The present application is related to U.S. patent application Ser. No. 11/680,471, filed on Feb. 28, 2007, now issued as U.S. Pat. No. 7,491,517, which is herein incorporated by reference in its entirety.

### FIELD

The present disclosure relates generally to the fields of medical microbiology, immunology, vaccines and the prevention of infection by a bacterial pathogen by immunization.

### BACKGROUND

Meningococcal meningitis is an infection of the meninges, which is a thin lining that surrounds the brain and the spinal cord. The causative agent, Neisseria meningitidis (also referred to as meningococcus), was identified in 1887. Meningococcal disease was first reported in 1805 when an outbreak swept through Geneva, Switzerland.

Thirteen subtypes or serogroups of N. meningitidis have been identified and six (N. meningitidis A, B, C, X, Y and W-135) are known to cause epidemics. Pathogenicity, immunogenicity, and epidemic capabilities differ according to the serogroup. Identification of the serogroup responsible for a sporadic case is important for epidemic containment. The most common symptoms are a stiff neck, high fever, and sensitivity to light, confusion, headaches, and vomiting. Even when the disease is diagnosed early and adequate therapy instituted, 5% to 10% of patients die, typically within 24-48 hours of the onset of symptoms. Bacterial meningitis may result in brain damage, hearing loss, or learning disability in 10 to 20% of survivors. A less common but more severe (and often fatal) form of meningococcal disease is meningococcal septicemia which is characterized by a hemorrhagic rash and rapid circulatory collapse.

Major African epidemics are associated with N. meningitidis serogroups A, W-135 and C, and serogroup A is usually the cause of meningococcal disease in Asia. Outside of Africa, Mongolia reported a large epidemic in 1994 to 1995. There is increasing evidence of serogroup W-135 being associated with outbreaks of considerable size. In 2000 and 2001 several hundred pilgrims attending the Hajj in Saudi Arabia were infected with N. meningitidis W-135. Then in 2002, W-135 emerged in Burkina Faso, striking 13,000 people and killing 1,500.

The highest burden of meningococcal disease occurs in sub-Saharan Africa, which is sometimes referred to as the “Meningitis Belt”, an area that stretches from Senegal in the west to Ethiopia in the east, with an estimated total population of 300 million people. This hyper-endemic area is characterized by a particular climate and social habits. During the dry season, between December and June, because of dust winds and upper respiratory tract infections due to cold nights, local immunity is diminished, increasing the risk of meningitis. At the same time, the transmission of N. meningitidis is favored by the overcrowded housing at the family level and by large population displacements due to pilgrimages and traditional markets at the regional level. This conjunction of factors contributes to the large epidemics which occur during this season in the meningitis belt area. Partly due to herd immunity (whereby transmission is blocked when a threshold percentage of the population has been vaccinated, which extends some protection to the unvaccinated), these epidemics tend to occur in a cyclic mode. N. meningitidis A, C, and W-135 are now the main serogroups involved in the meningococcal meningitis activity in Africa.

In 1996, Africa experienced the largest recorded outbreak of epidemic meningitis in history, with over 250,000 cases and 25,000 deaths registered. Between that crisis and 2002, 223,000 new cases of meningococcal meningitis were reported to the World Health Organization. The countries most affected have been Burkina Faso, Chad, Ethiopia, and Niger. In 2002, the outbreaks occurring in Burkina Faso, Ethiopia, and Niger accounted for about 65% of the total cases reported on the African continent. Furthermore, the meningitis belt appears to be extending further south. In 2002, the Great Lakes region was affected by outbreaks in villages and refugee camps which caused more than 2,200 cases, including 200 deaths.

In 2006 and 2007, outbreaks of the disease occurred in the North of Ivory Coast and the southern region of Burkina Faso, Southern Sudan, and Uganda, killing several children and adults. Meningococcal meningitis impacts not only Africa but also the rest of the world. Meningococcal meningitis impacts not only sub-Saharan Africa but also North America, the United Kingdom, Ireland, Europe, South East Asia, the Middle East, and New Zealand.

Currently, the capsular polysaccharides of Neisseria meningitidis have been considered as having highly conserved and highly exposed bacterial surface antigens. Currently, capsular polysaccharides have been used as immunoprophylactic agents against human disease caused by encapsulated bacteria. Currently, the capsular polysaccharides of the meningococcus are negatively charged and are obtained in a high-molecular-weight immunogenic form by precipitation. Currently, meningococcal polysaccharide vaccines are relatively efficacious for protection from meningitis disease in adults; however, the duration of protection elicited by existing meningococcal polysaccharide vaccines is not long lasting and has been estimated to be 18 months in adults and children above four years of age. Currently, for children from one to four years old, the duration of protection is less than three years.

Polysaccharides, themselves, have been found to be at least typically poor at stimulating an effective antibody response in the highest risk age groups (e.g., infants). Children less than two years of age are more susceptible to diseases caused by microbes that have polysaccharide capsules, such as Neisseria meningitidis. Current vaccines are expensive and have short duration of protection.

Discovery of a low-cost, more easily manufactured, and improved meningitis vaccine would be desirable for providing affordable vaccines to third world countries and would reduce mortality of infants, children, and adults. Additionally, there is a need for a method of producing a meningococcal meningitis vaccine at least substantially without chemical impurities or residues to improve depolymerization and conjugation by chemical means and capsular polysaccharide size. Also, there is a need for a medium that produces a higher yield of polysaccharides and a lower yield of cellular biomass to facilitate the production and purification processes for vaccine production.

### SUMMARY

In one aspect, embodiments of the inventive concepts disclosed herein are directed to a multivalent vaccine composition. The multivalent vaccine composition may include a mixture. The mixture may include conjugated Neisseria meningitides serogroup A oligosaccharides, where each of the conjugated Neisseria meningitides serogroup A oligosaccharides are conjugated to a first particular glycan-free carrier protein. The mixture may also include conjugated Neisseria meningitides serogroup C oligosaccharides, each of the conjugated Neisseria meningitides serogroup C oligosaccharides being conjugated to a second particular glycan-free carrier protein. The mixture may further include conjugated Neisseria meningitides serogroup Y oligosaccharides, each of the conjugated Neisseria meningitides serogroup Y oligosaccharides being conjugated to a third particular glycan-free carrier protein. The mixture may additionally include conjugated Neisseria meningitides serogroup W-135 oligosaccharides, each of the conjugated Neisseria meningitides serogroup W-135 oligosaccharides being conjugated to a fourth particular glycan-free carrier protein.

In a further aspect, embodiments of the inventive concepts disclosed herein are directed to method for producing a multivalent vaccine composition comprising Neisseria meningitides serogroups A, C, Y, and W-135 oligosaccharides conjugated to glycan-free carrier proteins. The method may include non-chemically adjusting sizes of Neisseria meningitides serogroups A, C, Y, and W-135 oligosaccharides. The method may include activating the Neisseria meningitides serogroups A, C, Y, and W-135 oligosaccharides upon non-chemically adjusting the sizes of the Neisseria meningitides serogroups A, C, Y, and W-135 oligosaccharides. The method may include activating glycan-free carrier proteins. The method may include conjugating at least some of the activated Neisseria meningitides serogroups A, C, Y, and W-135 oligosaccharides to at least some of the activated glycan-free carrier proteins. The method may include producing a multivalent vaccine composition comprising a mixture. The mixture may include conjugated Neisseria meningitides serogroup A oligosaccharides, each of the conjugated Neisseria meningitides serogroup A oligosaccharides being conjugated to a first particular glycan-free carrier protein of the activated glycan-free carrier proteins; conjugated Neisseria meningitides serogroup C oligosaccharides, each of the conjugated Neisseria meningitides serogroup C oligosaccharides being conjugated to a second particular glycan-free carrier protein of the activated glycan-free carrier proteins; conjugated Neisseria meningitides serogroup Y oligosaccharides, each of the conjugated Neisseria meningitides serogroup Y oligosaccharides being conjugated to a third particular glycan-free carrier protein of the activated glycan-free carrier proteins; and conjugated Neisseria meningitides serogroup W-135 oligosaccharides, each of the conjugated Neisseria meningitides serogroup W-135 oligosaccharides being conjugated to a fourth particular glycan-free carrier protein of the activated glycan-free carrier proteins

In a further aspect, embodiments of the inventive concepts disclosed herein are directed to an apparatus. The apparatus may include a vessel. The vessel may contain a multivalent vaccine composition. The multivalent vaccine composition may include a mixture. The mixture may include conjugated Neisseria meningitides serogroup A oligosaccharides, where each of the conjugated Neisseria meningitides serogroup A oligosaccharides being conjugated to a first particular glycan-free carrier protein. The mixture may also include conjugated Neisseria meningitides serogroup C oligosaccharides, each of the conjugated Neisseria meningitides serogroup C oligosaccharides being conjugated to a second particular glycan-free carrier protein. The mixture may further include conjugated Neisseria meningitides serogroup Y oligosaccharides, each of the conjugated Neisseria meningitides serogroup Y oligosaccharides being conjugated to a third particular glycan-free carrier protein. The mixture may additionally include conjugated Neisseria meningitides serogroup W-135 oligosaccharides, each of the conjugated Neisseria meningitides serogroup W-135 oligosaccharides being conjugated to a fourth particular glycan-free carrier protein.

In one aspect, embodiments of the inventive concepts disclosed herein are directed to a multivalent vaccine composition. The multivalent vaccine composition may include a mixture. The mixture may include a plurality of conjugated Neisseria meningitides serogroup oligosaccharides. The plurality of conjugated Neisseria meningitides serogroup oligosaccharides may include at least two serogroups of conjugated Neisseria meningitides serogroup A, C, Y, and W-135 oligosaccharides. Each of the plurality of conjugated Neisseria meningitides serogroup oligosaccharides being conjugated to a particular glycan-free carrier protein.

In one aspect, embodiments of the inventive concepts disclosed herein are directed to a vaccine composition. The multivalent vaccine composition may include a mixture. The mixture may include conjugated Neisseria meningitides serogroup oligosaccharides, each of the conjugated Neisseria meningitides serogroup oligosaccharides being conjugated to a particular glycan-free carrier protein.

### BRIEF DESCRIPTION OF THE DRAWINGS

Implementations of the inventive concepts disclosed herein may be better understood when consideration is given to the following detailed description thereof. Such description makes reference to the included drawings, which are not necessarily to scale, and in which some features may be exaggerated and some features may be omitted or may be represented schematically in the interest of clarity. Like reference numerals in the drawings may represent and refer to the same or similar element, feature, or function. In the drawings:

FIG. 1A is a view of an exemplary embodiment of a vessel containing a multivalent vaccine composition according to the inventive concepts disclosed herein.

FIG. 1B is a view of an exemplary embodiment of a vial containing a multivalent vaccine composition according to the inventive concepts disclosed herein.

FIG. 2 is a diagram of an exemplary embodiment of a method for producing a multivalent vaccine composition according to the inventive concepts disclosed herein.

### DETAILED DESCRIPTION

Before explaining at least one embodiment of the inventive concepts disclosed herein in detail, it is to be understood that the inventive concepts are not limited in their application to the details of construction and the arrangement of the components or steps or methodologies set forth in the following description or illustrated in the drawings. In the following detailed description of embodiments of the instant inventive concepts, numerous specific details are set forth in order to provide a more thorough understanding of the inventive concepts. However, it will be apparent to one of ordinary skill in the art having the benefit of the instant disclosure that the inventive concepts disclosed herein may be practiced without these specific details. In other instances, well-known features may not be described in detail to avoid unnecessarily complicating the instant disclosure. The inventive concepts disclosed herein are capable of other embodiments or of being practiced or carried out in various ways. Also, it is to be understood that the phraseology and terminology employed herein is for the purpose of description and should not be regarded as limiting.

As used herein a letter following a reference numeral is intended to reference an embodiment of the feature or element that may be similar, but not necessarily identical, to a previously described element or feature bearing the same reference numeral (e.g., 1, 1a, 1b). Such shorthand notations are used for purposes of convenience only, and should not be construed to limit the inventive concepts disclosed herein in any way unless expressly stated to the contrary.

Further, unless expressly stated to the contrary, “or” refers to an inclusive or and not to an exclusive or. For example, a condition A or B is satisfied by anyone of the following: A is true (or present) and B is false (or not present), A is false (or not present) and B is true (or present), and both A and B are true (or present).

In addition, use of the “a” or “an” are employed to describe elements and components of embodiments of the instant inventive concepts. This is done merely for convenience and to give a general sense of the inventive concepts, and “a” and “an” are intended to include one or at least one and the singular also includes the plural unless it is obvious that it is meant otherwise.

Finally, as used herein any reference to “one embodiment,” or “some embodiments” means that a particular element, feature, structure, or characteristic described in connection with the embodiment is included in at least one embodiment of the inventive concepts disclosed herein. The appearances of the phrase “in some embodiments” in various places in the specification are not necessarily all referring to the same embodiment, and embodiments of the inventive concepts disclosed may include one or more of the features expressly described or inherently present herein, or any combination of sub-combination of two or more such features, along with any other features which may not necessarily be expressly described or inherently present in the instant disclosure.

Broadly, embodiments of the inventive concepts disclosed herein are directed to a vaccine composition, a method for producing the vaccine composition, and an apparatus containing the vaccine composition.

Embodiments may include coupling T-cell independent saccharides to a T-cell dependent protein that allows an infant immune system to provide T-cell help to B-cells to produce IgG antibody of high affinity to the polysaccharide antigen. For example, T-Independent (TI) antigens may improve vaccines. Molecules, such as polysaccharides, that have numerous identical evenly spaced epitopes characterize one type of TI antigen. In one embodiment, as clusters of B-cell receptors bind the antigen simultaneously or approximately simultaneously, it causes B-cell activation without the help of T-helper cells. Such antigens may be particularly important in young children who typically respond poorly to these antigens.

In one embodiment, Neisseria meningitidis serogroup A, C, W-135 & Y Polysaccharide may be extracted from cultures grown in NMFM2 media in a fermenter vessel. For example, the cultures may be incubated at 36±1° C. up to a maximum of 24 hours while controlling temperature, pH, aeration, agitation, pressure, and dissolved oxygen content and antifoam additions whenever required. After the incubation period, a batch may be terminated when the glucose concentration reaches a threshold concentration (e.g., below 1 g/L), pH, and when dissolved oxygen content increases. The culture may be killed by the addition of formaldehyde; for example, 0.5% volume/volume concentration of formaldehyde may kill the culture. For example, adding about 420 mL of formaldehyde may kill 80 L of broth. Polysaccharide size adjustment may be achieved by a non-chemical method (e.g., sonication) to produce low molecular weight meningococcal oligosaccharides (e.g., with a minimum range of 5000 to 10,000 Daltons size). Sizes of oligosaccharides before and after sonication were compared by high-performance liquid chromatography-size exclusion chromatography (HPLC-SEC). In one embodiment, the carrier protein used in the conjugation with oligosaccharide is glycan-free diphtheria toxoid (GFDT), though other carrier proteins are contemplated. Glycan may be removed by Octyl-sepharose hydrophobic interaction chromatography and confirmed by high-performance anion exchange chromatography-pulsed amperometric detection analysis. In one embodiment, potluck oligosaccharide activation may be performed on multiple (e.g., 4) serogroup oligosaccharides that may be together activated by sodium periodate reaction to generate aldehyde active groups on them. For example, potluck oligosaccharide activation may be performed on 4 serogroup oligosaccharides that are together activated by 6 mM sodium periodate reaction at 22±2° C. for 4 hours to generate aldehyde active groups on them. After activation, the activated oligosaccharides may be diafiltered against HEPES using filters (e.g., membrane filters, such as MWCO membrane filters) to remove traces of sodium periodate. For example, after activation, the activated oligosaccharides are diafiltered against 10 mM HEPES pH7.5 at 2-8° C. for 15 h using 1K MWCO membrane filters to remove traces of sodium periodate. Oligosaccharide activation may be confirmed by colorimetric estimation of aldehyde groups using purpald assay. Acidic activation of diphtheria toxoid (DT) in the presence of EDC (3-ethyl-1-[3-dimethylamino) propyl] carbodiimide hydrochloride) and hydrazine may be used to create hydrazide groups on the DT. For example, acidic activation of the DT in the presence of EDC (3-ethyl-1-[3-dimethylamino) propyl] carbodiimide hydrochloride) hydrazine may be converted to hydrazide groups. Generated hydrazide groups on the toxoid may be quantified using colorimetric assay using trinitrobenzene sulfonic acid (TNBS) reagent where adipic acid dihydrazide (ADH) may be used as a standard in this assay. Protein content may be estimated using BCA assay kit following manufacturer's instructions. A degree of DT activation may be arrived at by measuring hydrazide groups per mole of DT by TNBS assay. For determination of DT degree of hydrazide activation measurement, an average molecular weight of DT may be taken as 63,000 Da. Embodiments may include conjugation of activated oligosaccharide to activated GFDT. For example, potluck activated oligosaccharides may be combined at suitable estimated concentration of each activated oligosaccharide (e.g., 20 mg/ml) and activated GFDT (e.g., 20 mg/ml) in a suitable weight ratio (e.g., 1:1), and mixture may be continuously mixed by rotating in suitable sealed containers, for example at 22±2° C. for 18-24 hours. The mixture may be treated with sodium borohydride, for example for 6 h at 22±2° C., to perform reductive amination and capping of unused aldehyde groups in a single step. Conjugate ultra-purification may be performed by filtration (e.g., 10K MWCO membrane filtration) to remove unconjugated low molecular weight oligosaccharide. For example, the conjugated and capped mixture may be diafiltered to remove unconjugated low molecular weight oligosaccharide using 10K MWCO membrane filters. Each oligosaccharide in the pure potluck conjugate may be quantified by HPAEC PAD analysis. With respect to the above potluck tetravalent conjugate, for example, the ultrapure mixture may be formulated in isotonic 1×PBS to contain a final concentration of 8 μg of each serogroup oligosaccharide conjugated to a total 32-64 μg glycan-free diphtheria Toxoid per each milliliter of formulated vaccine. For example, above formulated vaccine may be passed through 0.2μ membrane filters to obtain sterile final vaccine to consider for vialing.

An exemplary vaccine product was tested for safety and efficacy in 560 human volunteers comprising male and females of the ages 18 to 55. A safety study on the human subjects was observed for a total of 26 weeks for any safety concerns. No SAEs were observed in the phase 1 trial. Immunization with 4 μg of each serogroup oligosaccharide conjugated with 4-6 μg Glycan-free Diphtheria toxoid (per serogroup) produced seroconversion rates of 46-71% of subjects at 4 weeks: Serogroup A 46%, C 71%, W-135 63% and Y 67%. Higher seroconversion rates (71-92%) were observed at 8 weeks: A 71%, C 92%, W-135 67% and Y 75%.

Embodiments include a method of producing meningococcal meningitis vaccine comprising N. meningitidis serotypes A, C, Y and W-135 that have a long lasting effect and provide broad spectrum immunity to humans of all age groups.

Embodiments include a method wherein trace chemical impurities currently present in the available meningococcal meningitis vaccine are reduced (e.g., eliminated) by a mechanical method, such as sonication.

Embodiments include a composition of a medium that yields a higher percentage of polysaccharides in comparison to known media employed for producing meningococcal meningitis vaccine.

Embodiments include a composition of a medium that yields a lower percentage of cellular biomass in comparison with known media employed for producing meningococcal meningitis vaccine.

Embodiments include method of producing a vaccine composition and a vaccine composition having improved (e.g., optimum) molecular size of N. meningitidis polysaccharides of serogroups A, C, Y and W-135 that confers broad spectrum immunogenic protection against meningitis.

Embodiments include a multivalent (e.g., tetravalent) vaccine and methods of producing such a vaccine. Embodiments may include a meningococcal meningitis serogroups A, C, Y & W-135 vaccine made by using a fastidious culture medium (NMFM2). The downstream process may be animal component-free. Polysaccharide Size Adjustment may be performed by a non-chemical method (e.g., sonication) to produce low molecular weight polysaccharides and to produce conjugated meningococcal polysaccharides, for example, with a minimum range of 5000 to 10,000 Daltons size. A carrier protein used in the conjugation with oligosaccharide may be glycan-free diphtheria toxoid (GFDT). In an exemplary tetravalent embodiment, potluck oligosaccharide activation may be performed on at least some (e.g., all) of 4 serogroup oligosaccharides that may be together activated by (e.g., 6 mM) sodium periodate reaction. Activated oligosaccharides may be combined at estimated concentration of each activated oligosaccharide (e.g., 20 mg/ml) and activated GFDT (e.g., 20 mg/ml) in an exemplary weight ratio of approximately 1:1. For such a potluck tetravalent conjugate, a mixture (e.g., an ultrapure mixture) may be formulated in isotonic 1×PBS to contain a final concentration of 8 μg, for example, of each serogroup oligosaccharide conjugated to a total 32-64 μg, for example, glycan-free diphtheria Toxoid per each milliliter of formulated vaccine. Formulated vaccine may be sterilized at the stage of final fill.

Some embodiments may include a vaccine composition which does not include an adjuvant, but may include Glycan purified Diptheria Toxoid. Further, in some embodiments, the Diptheria Toxoid is not in a detoxified state. In some embodiments, the Glycan free Diptheria Toxoid is free from traces of Formaldehyde. Some embodiments include a hydrazide activated Glycan free Diptheria Toxoid. In some embodiments, the carrier protein of the vaccine composition is or includes a single Glycan free Diptheria Toxoid carrier protein species.

In some embodiments, the vaccine composition is formulated as an aseptic liquid. In some embodiments, the vaccine composition may be sterilized after formulation. In some embodiments, the vaccine composition is preservative-free. In some embodiments, the vaccine composition is a tetravalent conjugate vaccine comprising a mixture of four distinct and collective protein-capsular oligosaccharide conjugates of Serogroups A, C, Y and W-135 of Neisseria meningitidis.

In some embodiments, the capsular oligosaccharides have a minimum range of 5000 to 10,000 Daltons size of each of the serogroups depolymerized by non-chemical methods.

In some embodiments, the capsular oligosaccharide and Glycan-free Diptheria Toxoid is produced and processed with animal-component-free reagents and/or chemicals.

Trials were performed using ELISA bioassays because of transportation problems of live bacteria from the United States to Africa for performing SBA bioassays.

Meningococcal serogroup A, C, W-135, and Y polysaccharides and DT or CRM197-based conjugates may be prepared using suitable processes and conjugation chemistry. The polysaccharide content of serogroups C, W-135, and Y conjugates may be quantified by sialic acid determination. The Serogroup A conjugate may be quantified by mannosamine-1-phosphate chromatographic determination.

The protein content may be measured by a micro-bicinchoninic acid assay. For example, the polysaccharide-to-protein ratio of conjugates may have any suitable range, such as between 0.3 and 1.5, which may be similar to that of cross-reacting material DT and CRM-based conjugates.

A lymphocyte proliferation assay may be performed.

In addition, antigenic variation and human complement sensitivity of Neisseria meningitidis may be a barrier to relying on SBA bioassays.

Embodiments may include conjugation of bacterial polysaccharides to immunogenic carrier proteins that results in conjugates that induce strong anti-polysaccharide T-helper-cell dependent immune responses in young infants.

Neisseria meningitidis are gram negative diplococci with polysaccharide capsules that have led to the identification of at least 13 different serogroups based on immunochemical studies. Epidemics caused by N. meningitidis of Group A, C, Y, and W are usually characterized by a predominance of a single meningococcal genotype and a shift of cases towards older age groups. These four serogroups express structurally different capsular polysaccharides (shown below) that determine their distinct serological properties. O-acetylation of sugar units at distinct locations is seen in all these serogroups although at different levels.

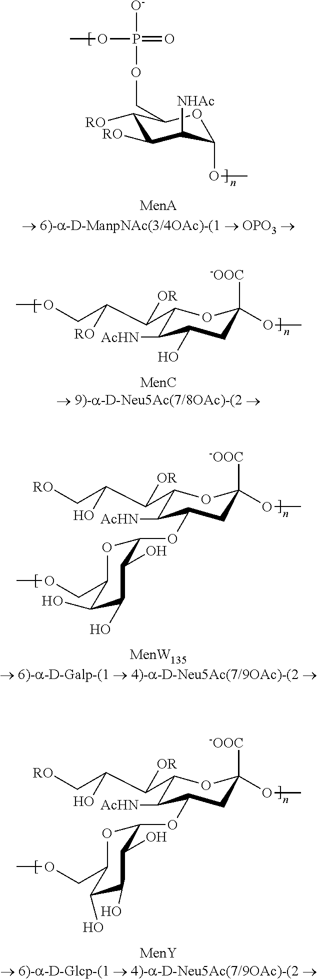

With respect to the above polysaccharide structures, the structural characterization and verification of vaccine grade polysaccharides may be performed by chemical and biophysical methods. NMR spectroscopy may be used for characterizing the meningococcal purified polysaccharides, and 1H NMR may be utilized for the estimation of the percentage O-acetylation in each serogroup. The O-acetyl transferases occurs in the serogroup A Manacc O-acetylation.

Group A isolates are now principally responsible for recurrent epidemics in the so called “meningitis belt” countries in sub-Saharan Africa. Existing Polysaccharide vaccines offer protection against groups A, C, Y, and W-135; however, such existing vaccines produce poor immune responses in young children and do not produce long-lasting immunity. Such existing vaccines utilize polysaccharides that are T-cell independent antigens such that they are poorly or not at all immunogenic in children aged less than 2 years, and such existing vaccines do not induce vaccine memory. Such existing vaccines use may predispose to blunting of the immune response to subsequent doses.

Embodiments may utilize chemical conjugation of the capsular polysaccharides to a protein carrier capable of eliciting T-cell dependent immune responses.

In an exemplary embodiment, a method of producing (e.g., preparing)
Meningococcal meningitis vaccine may include some or all of the following steps:
A, C, W-135 & Y Polysaccharide preparation;
Mechanical Size Adjustment of Polysaccharide;
Glycan- & Formalin-free Diphtheria Toxoid (GFDT);
Potluck Oligosaccharide Activation;
GFDT Activation;
Conjugation of Activated oligosaccharide to Activated GFDT to make individual Monoconjugate;
Conjugate ultra-purification by 10K MWCO membrane filtration;
Formulation of Tetravalent conjugates vaccine in 1×PBS;
Sterilization of final vaccine by 0.2-micron filtration; and
Vialing.

Such steps are exemplarily described, below.

A, C, W-135 & Y Polysaccharide Preparation:

In some embodiments, Neisseria meningitidis serogroup A, C, W135, and Y frozen seed cultures may be thawed and streaked onto NMFM2 Yeast extract meat free agar media. For example, such plates may be incubated at 36±1.0° C. with CO₂. 4±2% in the CO₂ incubator for 12 to 18 hours. Some embodiments may include inoculating 500 mL flasks containing 100 mL media with 2-3 lapful's of culture grown on the NMFM2 Yeast extract agar plates. Some embodiments may include incubating flasks at 36±1.0° C. on an incubator shaker at 170±30 RPM for 12±4 hours until OD₆₀₀ₙₘ reaches 2.5±1.0.

In some embodiments, once the flasks attain desired OD and pass Gram stain test, some embodiments may include pipetting 70 mL of inoculum into 630 mL media in 2 or 2.8 L flasks and incubating at 36±1° C. with 170±30 RPM on an incubator shaker for about 12±4 hours until the OD₆₀₀ₙₘ reaches in the range of 2.5±1.0.

The contents of the flasks may be transferred to a fermenter vessel containing sterile NMFM2 yeast extract medium. For example, the fermenter vessel may be incubated at 36±1° C. for up to a maximum of 24 hours with controlling temperature, pH, aeration, agitation, pressure, and dissolved oxygen content and antifoam additions whenever required. After the incubation period, the batch may be terminated when the glucose concentration reaches below 1 g/L, pH, and dissolved oxygen content increases. The culture may be killed by the addition of 0.5% v/v. Formaldehyde (e.g., add about 420 mL of Formaldehyde to kill 80 L broth).

Some embodiments may include holding the broth for about 1 hour to ensure that the culture was properly inactivated. Additionally, some embodiments may include setting the fermenter temperature between 10 to 20° C. before centrifuging the broth.

Some embodiments may include centrifuging the broth at around 15,000 to 20,000 g force with temperature of about 4° C. Some embodiments may include discarding the cell pellet and retaining the supernatant. Cetrimonium Bromide (CTAB) may be added to the supernatant to a final concentration of 0.1% to the 80 L supernatant (e.g., by adding 800 mL of 10% CTAB to make CTAB final concentration to 0.1%), and the material may be mixed for about 5 minutes and the contents may be left overnight (8-24 hours) for precipitation at room temperature (20-29° C.).

After overnight precipitation, some embodiments may include collecting the sedimented portion and discarding the supernatant. The precipitated CTAB portion may be centrifuged at around 15,000 to 20,000 g force with a temperature of about 4° C. (CTAB pellet can be kept in −80° C. until further processing).

Some embodiments may include suspending the CTAB pellet in 4 L of WFI or process water and stirring the contents for a minimum of 30 to 60 minutes at room temperature (20-29° C.) by using a magnetic stir bar and stirrer.

Some embodiments may include adding 4 L of 2M CaCl₂ to the CTAB pellet in WFI or process water, stirring the contents for about 30 to 60 minutes at 2-8° C., and ensuring that the final concentration of the mixture will be 1M CaCl₂.

Some embodiments may include, after the completion of stirring, centrifuging the above contents for 30 to 60 minutes at around 15,000 to 20,000 g force with a temperature of 4° C. Some embodiments may include retaining supernatant and discarding the pellet. Some embodiments may include adding 3200 mL of 200 proof ethanol to the above 8 L solution to make a final concentration of around 25% ethanol. (E.g., now, the volume will be 11.2 L).

Some embodiments may include mixing the calcium chloride wash with 25% ethanol and gently mixing for few minutes using a clean spatula. Some embodiments may include holding for 30-60 minutes at 2-8° C. and centrifuging the contents for about 30-60 minutes at around 15,000 to 20,000 g force with a temperature of 4° C. After centrifugation, some embodiments may include retaining supernatant and discarding the pellet. Some embodiments may include collecting the above supernatant and adding 3.4 to 4.0 (approximately 42 L to 50 L) volumes of 200 proof ethanol into clean 20 L glass bottles to make the contents reach around 80% ethanol concentration (+/−5% variation in ethanol concentration is acceptable). Some embodiments may include mixing the contents for about 5 minutes with a portable stirrer and then storing the contents 12-24 hours in a cold room at 2-8° C.

Some embodiments may include, after such 80% ethanol precipitation step, siphoning-off the top clear layer and discarding. Some embodiments may include collecting the precipitated portion and centrifuging the contents for about 30-60 minutes at around 15,000 to 20,000 g force with a temperature of 4° C. Some embodiments may include retaining the 80% ethanol pellet. The ethanol pellet can be stored in a −80° C. freezer until further processing. If the pellet is not stored in a −80° C. freezer, then some embodiments may include proceeding for pellet dissolution in water. For example, the 80% ethanol pellet can be processed in 1 or more parts.

Some embodiments may include, to the crude polysaccharide solution, adding enough Tris (e.g., pH 7.5 stock solution) and 1 M MgCl₂ stock solution to obtain a concentration of 20 mM Tris and 1 mM MgCl₂ and adding Benzonase at 1-5 μL per 100 mL of crude polysaccharide solution. Some embodiments may include incubating the contents in an incubator water bath shaker and shaking at 100 rpm at a temperature of 36±1° C. for about 12-16 hours. Some embodiments may include, to the above reaction mix, adding enough freshly prepared 5 mg/mL Proteinase K to obtain a final concentration of 50 μg/mL. Some embodiments may include continuing to mix at 100 RPM in the incubator-shaker at 36±1° C. for about 6-8 hours. Some embodiments may include, to the polysaccharide obtained in the above step, adding 1 M CaCl₂ of approximately 100 mL to 1 L of polysaccharide solution to obtain a final concentration of 0.1 M CaCl₂ and placing in an ultracentrifuge (e.g., at 100,000 g force) for 2 hours at 4° C. Some embodiments may include collecting the supernatant and discarding the pellet. Some embodiments may include proceeding with an ultrafiltration having a molecular weight cut-off of 100 membrane filters. If the above sample indicates non-permissive levels (>100 EU/μg of Polysaccharide) of endotoxin, some embodiments may include performing endotoxin removal using sartobind Q membranes to remove further endotoxin levels below 100 EU/μg of polysaccharide to ensure removal of endotoxin to a permissive level. Quality control passed material will be freeze dried under a vacuum and stored in a −80 degree Celsius freezer until further use.

Polysaccharide Size Adjustment:

Embodiments may include a non-chemical method (e.g., sonication) to make low molecular weight polysaccharides and to produce conjugated meningococcal polysaccharides with a minimum range of 5100 to 9900 Daltons size. Sizes of oligosaccharides before and after sonication sizes may be compared by HPLC-SEC (high-performance liquid chromatography-size exclusion chromatography).

Potluck Polysaccharide Activation:

In some embodiments, suitable amounts of four serogroup polysaccharides may be together activated by 6 mM sodium periodate reaction at 22±2° C. for 4 hours to generate aldehyde active groups on them. After the activation, the activated polysaccharides are diafiltered against 10 mM HEPES pH7.5 at 2-8° C. for 15 hours using 1K MWCO membrane filters to remove traces of sodium periodate. Oligosaccharide activation is confirmed by colorimetric estimation of aldehyde groups using purpald assay.

GFDT Activation:

During acidic activation of DT in the presence of EDC (3-ethyl-1-[3-dimethylamino) propyl] carbodiimide hydrochloride), hydrazine gets converted to hydrazide groups. In some embodiments, generated hydrazide groups on the toxoid are quantified using a colorimetric assay by using (TNBS) trinitrobenzene sulfonic acid reagent where adipic acid dihydrazide (ADH) is used as a standard in this assay.

In some embodiments, protein content is estimated using BCA assay kit following manufacturer's instructions.

In some embodiments, the degree of DT activation is arrived at by measuring hydrazide groups per mole of DT by TNBS assay. For determination of DT degree of hydrazide activation measurement, some embodiments may include an average molecular weight of DT taken as 63,000 Da (0.4 mM). For example, the below Table 1 shows a specification of 0.2-0.5 mN of hydrazide per mole of DT, i.e. with a 50-80% degree of activation.

TABLE 1

| Experminent | Hydrazide content | |
|---|---|---|
| No. | mN | Degree of activation % |
| 1 | 0.23 | 57.5 |
| 2 | 0.29 | 72.5 |
| 3 | 0.31 | 77.5 |
| 4 | 0.30 | 75.0 |

Conjugation of Activated Oligosaccharide to Activated GFDT Potluck-Conjugates:

For example, potluck activated oligosaccharides are combined at estimated concentration of each activated oligosaccharide (20 mg/ml) and activated GFDT (20 mg/ml) in the weight ratio of 1:1, and such mixture is continuously mixed by rotating in suitable sealed containers at 22±2° C. for 18-24 hours. The resulting mixture may then be treated with sodium borohydride for 6 h at 22±2° C. to perform reductive amination and capping of unused aldehyde groups in a single step.

Conjugate Ultra-Purification by 10K MWCO Membrane Filtration:

For example, the above conjugated and capped mixture is diafiltered to remove unconjugated low molecular weight oligosaccharide using 10K MWCO membrane filters. Quantification of each Oligosaccharide in the pure potluck conjugate may be performed by HPAEC PAD analysis.

Formulation of Tetravalent Conjugate Vaccine in 1×PBS:

In some embodiments, the above potluck tetravalent conjugate ultrapure mixture is formulated in isotonic 1×PBS to contain a final concentration of 8 μg of each serogroup oligosaccharide conjugated to a total 32-64 μg diphtheria Toxoid per each milliliter of formulated vaccine.

Sterilization of final vaccine by 0.2-micron filtration: For example, the above formulated vaccine may be passed through 0.2μ membrane filters to obtain a sterile final vaccine to consider for vialing.

Phase 1 Study: A Double-Blind, Randomized, Controlled, Two Arm Phase 1 Clinical Trial of the Safety and Immunogenicity of Group A, C, Y & W-135 Meningococcal Polysaccharide DT Conjugate Vaccine: NmVac4-A/C/Y/W-135-DT™.

Clinical Trail Study Location: Hanford, Calif., USA

Our previous pre-clinical experience and the experience of others with similar vaccines which are already in the market, suggests that one dose of Meningococcal meningitis A, C, Y & W-135 polysaccharide diphtheria conjugate vaccine is likely to show satisfactory safety and induce a protective immune response in naive individuals.

Safety Results:

In our Phase, 1 Safety Study, 60 age 18-50-year-old subjects (30 test vaccine, 30 active control vaccine) were observed for a total of 26 weeks for any safety concerns. No SAEs were observed in the phase 1 trial. Adverse event severity and frequency were similar to those reported for published meningococcal vaccine studies. Treatment-related adverse events observed in our phase 1 study are shown in Table 1. Most were mild or moderate. Two Grade 3 AEs (a headache and anorexia) were possibly associated with the vaccine. The subjects were fully recovered within 1 week of vaccination.

TABLE 2
Summary of Treatment-Related Adverse Events

| System Organ Class | | Subject Count (%) by Strata | | | |
|---|---|---|---|---|---|
| MedDRA Preferred | | | | Control | Control |
| Term | Total | NmVac4- | NmVac4- | vaccine/ | vaccine/ |
| (sorted by | Subject | DT/Female | DT/Male | Female | Male |
| frequency) | Count (%) | (N = 15) | (N = 15) | (N = 15) | (N = 15) |
| Any adverse event | 32 (53%) | 7 (47%) | 9 (60%) | 9 (60%) | 7 (47%) |
| General Disorders: | 27 (45%) | 6 (40%) | 8 (60%) | 7 (40%) | 6 (40%) |
| Administration site | | | | | |
| conditions | | | | | |
| General disorders: | 9 (15%) | 0 (0%) | 3 (20%) | 3 (20%) | 3 (20%) |
| Chills, Fatigue, | | | | | |
| Malaise | | | | | |
| Gastrointestinal | 9 (15%) | 3 (20%) | 3 (20%) | 3 (20%) | 0 (0%) |
| disorders: nausea, | | | | | |
| diarrhea | | | | | |
| Musculoskeletal and | 8 (13%) | 2 (13%) | 3 (20%) | 2 (13%) | 1 (7%) |
| connective tissue | | | | | |
| disorders | | | | | |
| Nervous system | 8 (13%) | 1 (7%) | 3 (20%) | 2 (13%) | 2 (13%) |
| disorders: Headache | | | | | |
| Metabolism and | 2 (3.3%) | 0 (0%) | 1 (7%) | 1 (7%) | 0 (0%) |
| nutrition disorders: | | | | | |
| Anorexia | | | | | |
| Vital Signs | 1 (1.7%) | 0 (0%) | 0 (0%) | 0 (0%) | 1 (7%) |

Immunogenicity Results:

Immunization with 4 μg of each serogroup oligosaccharide conjugated with 4-6 μg GF Diphtheria toxoid (per serogroup) produced seroconversion rates (by rSBA) of 46-71% of subjects at 4 weeks: Serogroup A 46%, C 71%, W-135 63% and Y 67%. Higher seroconversion rates (71-92%) were observed at 8 weeks: A 71%, C 92%, W-135 67% and Y 75%. Immunogenicity results were similar for control and test vaccines (Table 2). These preliminary results indicate that a single dose may be sufficient to protect adult subjects from Meningococcal disease and may be useful for endemic seasonal immunization as an effective response to the global need for vaccination against this pandemic and endemic disease.

TABLE 3
Seroconversion Rate and GMT in the Per Protocol Population by
Treatment.

| | Sero- | | NmVac4-DT | Control vaccine |
|---|---|---|---|---|
| Variable | group | Visit | (N = 24) | (N = 25) |
| Seroconversion | A | Week 4 | 46% (26%, 67%) | 68% (46%, 85%) |
| % (95% CI) | | | | |
| GMT (95% CI) | A | Week 4 | 12.7 (10.4, 33.0) | 12.8 (11.3, 31.7) |
| Seroconversion | A | Week 8 | 71% (49%, 87%) | 80% (59%, 93%) |
| % (95% CI) | | | | |
| GMT (95% CI) | A | Week 8 | 13.0 (12.0, 33.8) | 15.8 (20.8, 68.7) |
| Seroconversion | C | Week 4 | 71% (49%, 87%) | 80% (59%, 93%) |
| % (95% CI) | | | | |
| GMT (95% CI) | C | Week 4 | 18.5 (10.4, 33.0) | 18.9 (11.3, 31.7) |
| Seroconversion | C | Week 8 | 92% (73%, 99%) | 68% (46%, 85%) |
| % (95% CI) | | | | |
| GMT (95% CI) | C | Week 8 | 22.6 (14.6, 35.1) | 19.4 (12.5, 30.2) |
| Seroconversion | W-135 | Week 4 | 63% (41%, 81%) | 76% (55%, 91%) |
| % (95% CI) | | | | |
| GMT (95% CI) | W-135 | Week 4 | 18.0 (9.1, 35.6) | 29.4 (14.4, 60.0) |
| Seroconversion | W-135 | Week 8 | 67% (45%, 84%) | 68% (46%, 85%) |
| % (95% CI) | | | | |
| GMT (95% CI) | W-135 | Week 8 | 19.6 (11.3, 33.9) | 24.3 (12.9, 45.5) |
| Seroconversion | Y | Week 4 | 67% (45%, 84%) | 72% (51%, 88%) |
| % (95% CI) | | | | |
| GMT (95% CI) | Y | Week 4 | 22.0 (12.6, 38.5) | 31.1 (16.7, 57.9) |
| Seroconversion | Y | Week 8 | 75% (53%, 90%) | 60% (39%, 79%) |
| % (95% CI) | | | | |
| GMT (95% CI) | Y | Week 8 | 19.0 (11.3, 32.0) | 21.7 (11.9, 39.6) |

Some embodiments may include at least one processor configured to run various software applications or computer code stored in a non-transitory computer-readable medium and configured to execute various instructions or operations, including but not limited to instructions or operations associated with processes, systems, equipment, functions, and methods disclosed throughout. The at least one processor may be configured to run various software applications or computer code stored in a non-transitory computer-readable medium and configured to execute various instructions or operations as disclosed throughout and configured to perform any suitable functions. The at least one processor may be implemented in any number (e.g., at least one, two, or more) of computing devices that may or may not be interconnected over a network (e.g., a local area network (LAN), wireless area network (WAN), the Internet, or a combination thereof) and/or communicatively coupled (e.g., via a network) with other computing devices. Such computing devices may include at least one processor, memory, storage, or the like, and such computing devices may include or be communicatively coupled to any of various suitable sensors configured to measure properties (e.g., pressure, gas composition, spectrometer, etc.) associated with systems and processes disclosed throughout.

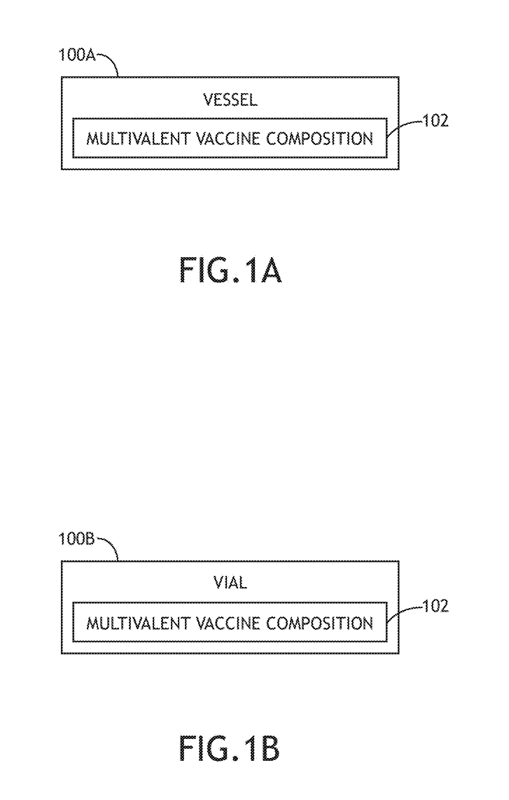

Referring now to FIG. 1A, an exemplary embodiment of a vessel 100A containing a multivalent vaccine composition 102 according to the inventive concepts disclosed herein is depicted. The vessel 100A may be or may be implemented as any vessel (e.g., a container, a vial (e.g., vial 100B, as shown in FIG. 1B), or a syringe chamber) suitable for containing the multivalent vaccine composition 102.

In some embodiments, the multivalent vaccine composition 102 may comprise a vaccine composition including two or more Neisseria meningitides serogroup oligosaccharides conjugated to glycan-free carrier proteins. For example, the multivalent vaccine composition 102 may include a liquid mixture including Neisseria meningitides serogroups A, C, Y, and W-135 oligosaccharides conjugated to glycan-free carrier proteins. For example, the multivalent vaccine composition 102 may include a mixture of conjugated Neisseria meningitides serogroup A, C, Y, and W-135 oligosaccharides, where each of the conjugated Neisseria meningitides serogroup A, C, Y, and W-135 oligosaccharides is conjugated to a glycan-free carrier protein. For example, the multivalent vaccine composition may include a mixture of the following: a) conjugated Neisseria meningitides serogroup A oligosaccharides, each of the conjugated Neisseria meningitides serogroup A oligosaccharides being conjugated to a first particular glycan-free carrier protein; b) conjugated Neisseria meningitides serogroup C oligosaccharides, each of the conjugated Neisseria meningitides serogroup C oligosaccharides being conjugated to a second particular glycan-free carrier protein; c) conjugated Neisseria meningitides serogroup Y oligosaccharides, each of the conjugated Neisseria meningitides serogroup Y oligosaccharides being conjugated to a third particular glycan-free carrier protein; and d) conjugated Neisseria meningitides serogroup W-135 oligosaccharides, each of the conjugated Neisseria meningitides serogroup W-135 oligosaccharides being conjugated to a fourth particular glycan-free carrier protein.

When administered, the multivalent vaccine composition 102 may provide long-lasting immunity against two or more serogroups (e.g., four, such as serogroups A, C, Y, and W-135) of N. meningitidis to humans of all age groups, including infants.

While the vessel 100A exemplarily includes elements as shown, in some embodiments, the vessel 100A may include other elements.

Referring now to FIG. 1B, an exemplary embodiment of a vial 100B containing the multivalent vaccine composition 102 according to the inventive concepts disclosed herein is depicted. The vial 100B containing the multivalent vaccine composition 102 may be implemented similarly to the vessel 100A containing a multivalent vaccine composition 102.

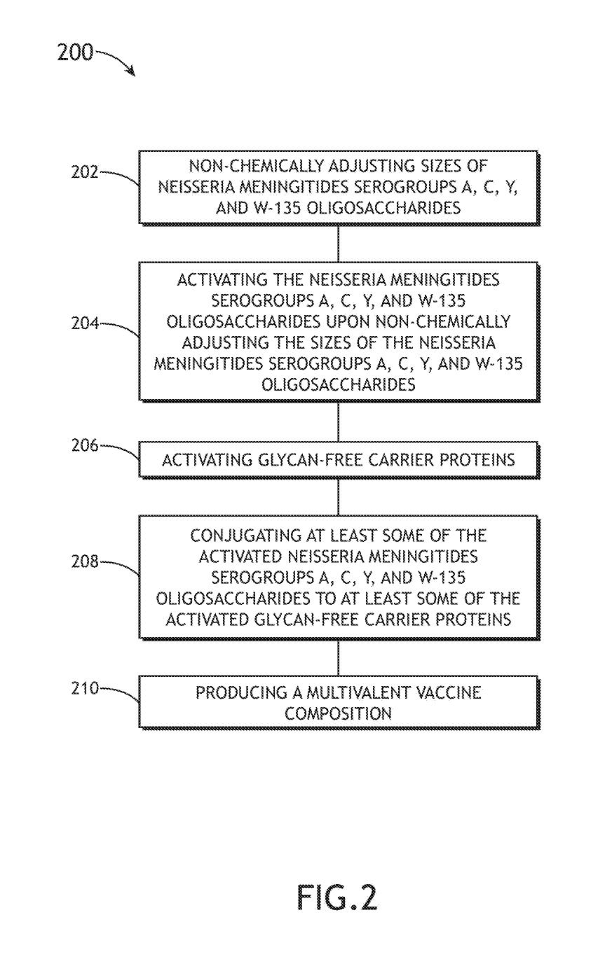

Referring now to FIG. 2, an exemplary embodiment of a method 200 for method for producing a multivalent vaccine composition according to the inventive concepts disclosed herein may include one or more of the following steps, which, for example, may be controlled and/or performed by any suitable components disclosed throughout. Additionally, for example, some embodiments may include performing and/or controlling one more instances of the method 200 iteratively, concurrently, and/or sequentially.

A step 202 may include non-chemically adjusting sizes of Neisseria meningitides serogroups A, C, Y, and W-135 oligosaccharides.

A step 204 may include activating the Neisseria meningitides serogroups A, C, Y, and W-135 oligosaccharides upon non-chemically adjusting the sizes of the Neisseria meningitides serogroups A, C, Y, and W-135 oligosaccharides.

A step 206 may include activating glycan-free carrier proteins.

A step 208 may include conjugating at least some of the activated Neisseria meningitides serogroups A, C, Y, and W-135 oligosaccharides to at least some of the activated glycan-free carrier proteins.

A step 210 may include producing a multivalent vaccine composition including a mixture. The mixture may include one or more (e.g., one, two, three, or four) of the following: a) conjugated Neisseria meningitides serogroup A oligosaccharides, each of the conjugated Neisseria meningitides serogroup A oligosaccharides being conjugated to a first particular glycan-free carrier protein of the activated glycan-free carrier proteins; b) conjugated Neisseria meningitides serogroup C oligosaccharides, each of the conjugated Neisseria meningitides serogroup C oligosaccharides being conjugated to a second particular glycan-free carrier protein of the activated glycan-free carrier proteins; c) conjugated Neisseria meningitides serogroup Y oligosaccharides, each of the conjugated Neisseria meningitides serogroup Y oligosaccharides being conjugated to a third particular glycan-free carrier protein of the activated glycan-free carrier proteins; and d) conjugated Neisseria meningitides serogroup W-135 oligosaccharides, each of the conjugated Neisseria meningitides serogroup W-135 oligosaccharides being conjugated to a fourth particular glycan-free carrier protein of the activated glycan-free carrier proteins.

Further, the method 200 may include any of the operations, steps, and/or actions disclosed throughout. For example, the method 200 may include removing unconjugated oligosaccharides from a mixture including the activated A, C, Y, and W-135 oligosaccharides conjugated to the glycan-free carrier protein. Further, for example, the method 200 may include sterilizing a mixture including the activated A, C, Y, and W-135 oligosaccharides conjugated to the glycan-free carrier protein. Also, for example, the method 200 may include vialing at least a portion of the multivalent vaccine composition comprising Neisseria meningitides serogroups A, C, Y, and W-135 oligosaccharides conjugated to a glycan-free carrier protein in a vessel.

As will be appreciated from the above, embodiments of the inventive concepts disclosed herein may be directed to a vaccine composition, a method for producing the vaccine composition, and an apparatus containing the vaccine composition.

As used throughout and as would be appreciated by those skilled in the art, “at least one non-transitory computer-readable medium” may refer to as at least one non-transitory computer-readable medium (e.g., memory, storage, or a combination thereof; e.g., at least one computer-readable medium implemented as hardware; e.g., at least one non-transitory processor-readable medium, at least one memory (e.g., at least one nonvolatile memory, at least one volatile memory, or a combination thereof; e.g., at least one random-access memory, at least one flash memory, at least one read-only memory (ROM) (e.g., at least one electrically erasable programmable ROM (EEPROM), at least one on-processor memory (e.g., at least one on-processor cache, at least one on-processor buffer, at least one on-processor flash memory, at least one on-processor EEPROM, or a combination thereof), or a combination thereof), at least one storage device (e.g., at least one hard-disk drive, at least one tape drive, at least one solid-state drive, at least one flash drive, at least one readable and/or writable disk of at least one optical drive configured to read from and/or write to the at least one readable and/or writable disk, or a combination thereof), or a combination thereof.

As used throughout, “at least one” means one or a plurality of; for example, “at least one” may comprise one, two, three, . . . , one hundred, or more. Similarly, as used throughout, “one or more” means one or a plurality of; for example, “one or more” may comprise one, two, three, . . . , one hundred, or more. Further, as used throughout, “zero or more” means zero, one, or a plurality of; for example, “zero or more” may comprise zero, one, two, three, . . . , one hundred, or more.

In the present disclosure, the methods, operations, and/or functionality disclosed may be implemented as sets of instructions or software readable by a device (e.g., a processor of a computing device or a controller). Further, it is understood that the specific order or hierarchy of steps in the methods, operations, and/or functionality disclosed are examples of exemplary approaches. Based upon design preferences, it is understood that the specific order or hierarchy of steps in the methods, operations, and/or functionality can be rearranged while remaining within the scope of the inventive concepts disclosed herein. The accompanying claims may present elements of the various steps in a sample order, and are not necessarily meant to be limited to the specific order or hierarchy presented.

It is to be understood that embodiments of the methods according to the inventive concepts disclosed herein may include one or more of the steps described herein. Further, such steps may be carried out in any desired order and two or more of the steps may be carried out simultaneously with one another. Two or more of the steps disclosed herein may be combined in a single step, and in some embodiments, one or more of the steps may be carried out as two or more sub-steps. Further, other steps or sub-steps may be carried in addition to, or as substitutes to one or more of the steps disclosed herein.

From the above description, it is clear that the inventive concepts disclosed herein are well adapted to carry out the objects and to attain the advantages mentioned herein as well as those inherent in the inventive concepts disclosed herein. While presently preferred embodiments of the inventive concepts disclosed herein have been described for purposes of this disclosure, it will be understood that numerous changes may be made which will readily suggest themselves to those skilled in the art and which are accomplished within the broad scope and coverage of the inventive concepts disclosed and claimed herein.

What is claimed is:

1. A multivalent vaccine composition, comprising:
a mixture comprising:
conjugated Neisseria meningitides serogroup A oligosaccharides, each of the conjugated Neisseria meningitides serogroup A oligosaccharides being conjugated to a first particular glycan-free carrier protein;
conjugated Neisseria meningitides serogroup C oligosaccharides, each of the conjugated Neisseria meningitides serogroup C oligosaccharides being conjugated to a second particular glycan-free carrier protein;
conjugated Neisseria meningitides serogroup Y oligosaccharides, each of the conjugated Neisseria meningitides serogroup Y oligosaccharides being conjugated to a third particular glycan-free carrier protein; and
conjugated Neisseria meningitides serogroup W-135 oligosaccharides, each of the conjugated Neisseria meningitides serogroup W-135 oligosaccharides being conjugated to a fourth particular glycan-free carrier protein.

2. The multivalent vaccine composition of claim 1, wherein at least one of the first, second, third, and fourth particular glycan-free carrier proteins is a glycan-free diphtheria toxoid.

3. The multivalent vaccine composition of claim 2, wherein the conjugated Neisseria meningitides serogroup A, C, Y, and W-135 oligosaccharides of the mixture have sizes approximately between 5100 and 9900 Daltons.

4. The multivalent vaccine composition of claim 3, wherein the first, second, third, and fourth particular glycan-free carrier proteins are glycan-free diphtheria toxoid,
wherein, with respect to at least one of the conjugated Neisseria meningitides serogroup A, C, Y, and W-135 oligosaccharides of the multivalent vaccine composition, a ratio of a mass of unconjugated Neisseria meningitides serogroup oligosaccharides to a mass of glycan-free diphtheria toxoid is approximately between 1:1 and 1:2.

5. The multivalent vaccine composition of claim 3, wherein the multivalent vaccine composition does not include an adjuvant.

6. The multivalent vaccine composition of claim 5, wherein the glycan-free diphtheria toxoid is not in a detoxified state.

7. The multivalent vaccine composition of claim 6, wherein the glycan-free diphtheria toxoid is glycan-free and formalin-free diphtheria toxoid.

8. The multivalent vaccine composition of claim 6, wherein the glycan-free diphtheria toxoid is hydrazide-activated glycan-free diphtheria toxoid.

9. The multivalent vaccine composition of claim 1, wherein each of the first, second, third, and fourth particular glycan-free carrier protein is of a single glycan-free diphtheria toxoid species.

10. The multivalent vaccine composition of claim 1, wherein the multivalent vaccine composition is formulated as an aseptic liquid and is sterilized after formulation.

11. The multivalent vaccine composition of claim 10, wherein the multivalent vaccine composition contains no preservative.

12. The multivalent vaccine composition of claim 1, wherein the multivalent vaccine composition is produced and processed by utilizing animal-component-free reagents and chemicals.

13. The multivalent vaccine composition of claim 1, wherein at least one of the first, second, third, and fourth particular glycan-free carrier proteins is glycan-free and formalin-free diphtheria toxoid.

14. The multivalent vaccine composition of claim 1, wherein the multivalent vaccine composition is a tetravalent vaccine composition.

15. The multivalent vaccine composition of claim 1, wherein the multivalent vaccine composition is a tetravalent vaccine composition and each of the first, second, third, and fourth particular glycan-free carrier proteins is glycan-free and formalin-free diphtheria toxoid.

16. The multivalent vaccine composition of claim 1, wherein the mixture comprises four distinct and collective protein-capsular oligosaccharide monoconjugates of serogroups A, C, Y, and W-135 of Neisseria meningitides.

17. The multivalent vaccine composition of claim 1, wherein the conjugated Neisseria meningitides serogroup A, C, Y, and W-135 oligosaccharides of the mixture have sizes approximately between 5100 and 9900 Daltons.

18. The multivalent vaccine composition of claim 17, wherein the multivalent vaccine composition is configured to provide immunity to humans of all age groups for at least three years.

19. The multivalent vaccine composition of claim 17, wherein the multivalent vaccine composition is configured to provide immunity to infants.

20. The multivalent vaccine composition of claim 17, wherein the multivalent vaccine composition is configured to provide immunity to children less than 2 years of age for at least three years.

21. The multivalent vaccine composition of claim 1, wherein the multivalent vaccine composition is configured to provide immunity to humans of all age groups for at least three years.

22. The multivalent vaccine composition of claim 1, wherein the multivalent vaccine composition is configured to provide immunity to infants.

23. The multivalent vaccine composition of claim 1, wherein the multivalent vaccine composition is configured to provide immunity to children less than 2 years of age for at least three years.

24. A method for producing a multivalent vaccine composition comprising Neisseria meningitides serogroups A, C, Y, and W-135 oligosaccharides conjugated to glycan-free carrier proteins, the method comprising:
non-chemically adjusting sizes of Neisseria meningitides serogroups A, C, Y, and W-135 oligosaccharides;
activating the Neisseria meningitides serogroups A, C, Y, and W-135 oligosaccharides upon non-chemically adjusting the sizes of the Neisseria meningitides serogroups A, C, Y, and W-135 oligosaccharides;
activating glycan-free carrier proteins;
conjugating at least some of the activated Neisseria meningitides serogroups A, C, Y, and W-135 oligosaccharides to at least some of the activated glycan-free carrier proteins; and
producing a multivalent vaccine composition comprising a mixture, the mixture comprising:
conjugated Neisseria meningitides serogroup A oligosaccharides, each of the conjugated Neisseria meningitides serogroup A oligosaccharides being conjugated to a first particular glycan-free carrier protein of the activated glycan-free carrier proteins;
conjugated Neisseria meningitides serogroup C oligosaccharides, each of the conjugated Neisseria meningitides serogroup C oligosaccharides being conjugated to a second particular glycan-free carrier protein of the activated glycan-free carrier proteins;
conjugated Neisseria meningitides serogroup Y oligosaccharides, each of the conjugated Neisseria meningitides serogroup Y oligosaccharides being conjugated to a third particular glycan-free carrier protein of the activated glycan-free carrier proteins; and
conjugated Neisseria meningitides serogroup W-135 oligosaccharides, each of the conjugated Neisseria meningitides serogroup W-135 oligosaccharides being conjugated to a fourth particular glycan-free carrier protein of the activated glycan-free carrier proteins.

25. The method of claim 24, wherein the conjugated A, C, Y, and W-135 oligosaccharides have sizes approximately between 5100 and 9900 Daltons.

26. The method of claim 24, further comprising:
removing unconjugated oligosaccharides from a mixture including the activated A, C, Y, and W-135 oligosaccharides conjugated to the glycan-free carrier proteins.

27. The method of claim 24, further comprising:
sterilizing a mixture including the activated A, C, Y, and W-135 oligosaccharides conjugated to the glycan-free carrier proteins.

28. The method of claim 24, further comprising:
vialing at least a portion of the multivalent vaccine composition comprising Neisseria meningitides serogroups A, C, Y, and W-135 oligosaccharides conjugated to the glycan-free carrier proteins.

29. The method of claim 24, wherein the glycan-free carrier proteins are glycan-free and formalin-free diphtheria toxoid.

30. The method of claim 24, wherein the multivalent vaccine composition is a tetravalent vaccine composition.

31. The method of claim 24, wherein the glycan-free carrier proteins are glycan-free diphtheria toxoid.

32. An apparatus, comprising:
a vessel containing a multivalent vaccine composition, the multivalent vaccine composition comprising:
a mixture comprising:
conjugated Neisseria meningitides serogroup A oligosaccharides, each of the conjugated Neisseria meningitides serogroup A oligosaccharides being conjugated to a first particular glycan-free carrier protein;
conjugated Neisseria meningitides serogroup C oligosaccharides, each of the conjugated Neisseria meningitides serogroup C oligosaccharides being conjugated to a second particular glycan-free carrier protein;
conjugated Neisseria meningitides serogroup Y oligosaccharides, each of the conjugated Neisseria meningitides serogroup Y oligosaccharides being conjugated to a third particular glycan-free carrier protein; and
conjugated Neisseria meningitides serogroup W-135 oligosaccharides, each of the conjugated Neisseria meningitides serogroup W-135 oligosaccharides being conjugated to a fourth particular glycan-free carrier protein.

33. The apparatus of claim 32, wherein the vessel is a vial.

34. A multivalent vaccine composition, comprising:
a mixture comprising:
a plurality of conjugated Neisseria meningitides serogroup oligosaccharides, the plurality of conjugated Neisseria meningitides serogroup oligosaccharides including at least two serogroups of conjugated Neisseria meningitides serogroup A, C, Y, and W-135 oligosaccharides, each of the plurality of conjugated Neisseria meningitides serogroup oligosaccharides being conjugated to a particular glycan-free carrier protein.

35. The multivalent vaccine composition of claim 34, wherein the plurality of conjugated Neisseria meningitides serogroup oligosaccharides including at least three serogroups of conjugated Neisseria meningitides serogroup A, C, Y, and W-135 oligosaccharides.

36. A vaccine composition, comprising:
a mixture comprising:
conjugated Neisseria meningitides serogroup oligosaccharides, each of the conjugated Neisseria meningitides serogroup oligosaccharides being conjugated to a particular glycan-free carrier protein.
